(19) United States
(12) Patent Application Publication
(10) Pub. No.: US 2007/0293886 A1
(43) Pub. Date: Dec. 20, 2007

(54) Suppository for hemorrhoid treatment

(76) Inventor: Jiro Takashima, Houston, TX (US)

Correspondence Address: EGBERT LAW OFFICES, 412 MAIN STREET, 7TH FLOOR, HOUSTON, TX, 77002, US

(21) Appl. No.: 11/452,628

(22) Filed: Jun. 15, 2006

(51) Int. Cl. A61M 31/00

(52) U.S. Cl. 606/197; 604/48

(57) ABSTRACT

A suppository for use in an anal canal has a shaft and ring positioned on the shaft between the first end and the second end of the shaft. The ring extends outwardly of the shaft and has a diameter of between 8 and 14 millimeters. The ring has a generally toroidal shape. The shaft and the ring are preferably integrally formed together. The diameter of the shaft is, preferably, between ⅓ and ⅔ of a maximum diameter of the ring.

### RELATED U.S. APPLICATIONS

Not applicable.

### STATEMENT REGARDING FEDERALLY SPONSORED RESEARCH OR DEVELOPMENT

Not applicable.

### Reference to Microfiche Appendix

Not applicable.

### FIELD OF THE INVENTION

The present invention relates to hemorrhoid treatment devices. More particularly, the present invention relates to suppositories as used for such hemorrhoid treatment. Additionally, the present invention relates to hemorrhoid suppositories that are designed so as to stay within the anal canal in order to deliver the medication directly to the internal hemorrhoidal area.

### BACKGROUND OF THE INVENTION

Most hemorrhoids have a bullet shape. It is quite difficult for these bullet-shaped suppositories to stay within the anal canal after they are inserted. Additionally, these bullet-shaped suppositories tend to go upwardly in the rectum before the medication is directly applied to the internal hemorrhoidal area.

It is an object of the present invention to provide a suppository which allows the suppository to be movably retained within the anal canal by utilizing the friction between the suppository and the anal canal.

It is another object of the present invention to provide a suppository for hemorrhoid treatment which is retained naturally within the anal canal so as to avoid entering the rectum or being pushed outside of the anus.

It is another object of the present invention to provide a suppository that particularly adapted to the size and structure of the anal canal.

It is further object of the present invention to provide a suppository that is easy to use, easy to manufacture and relatively inexpensive.

These and other objects and advantages of the present invention will become apparent from a reading of the attached specification and appended claims.

### BRIEF SUMMARY OF THE INVENTION

The present invention is a suppository for use in an anal canal that comprises a shaft having a first end and second end, and a ring formed on the shaft between the first end and the second end. The shaft and the ring are preferably formed of a material that is dissolvable when received within the anal canal. The ring extends outwardly of the shaft and has a diameter greater than the diameter of the shaft.

In the present invention, the shaft has a generally constant diameter between the first end and the second end. In particular, the shaft has a length of no less than 3 centimeters. Each of the first and second ends of the shaft are dome-shaped. The minimum length of the wide portion of the ring to either end of the shaft is approximately 2 centimeters.

The ring is preferably formed generally centrally along the shaft between the first and second ends. The ring extends around the outer diameter of the shaft. This ring has in the preferred embodiment, a generally toroidal shape. The ring will have an optimum diameter of between 6 and 12 millimeters inclusive. Additionally, the ring has a thickness of no less than the diameter of the wide portion of the ring.

The shaft and the ring are integrally formed together in the preferred embodiment. The diameter of the shaft is between ⅓ and ⅔ of the diameter of the ring.

### BRIEF DESCRIPTION OF THE SEVERAL VIEWS OF THE DRAWINGS

FIG. 1 is a side elevational view of the suppository of the present invention.

FIG. 2 is a diagrammatic illustration of the suppository of the present invention as located within anal canal.

FIG. 3 is a bottom view of the suppositories of the present invention as illustrated in FIG. 1.

### DETAILED DESCRIPTION OF THE INVENTION

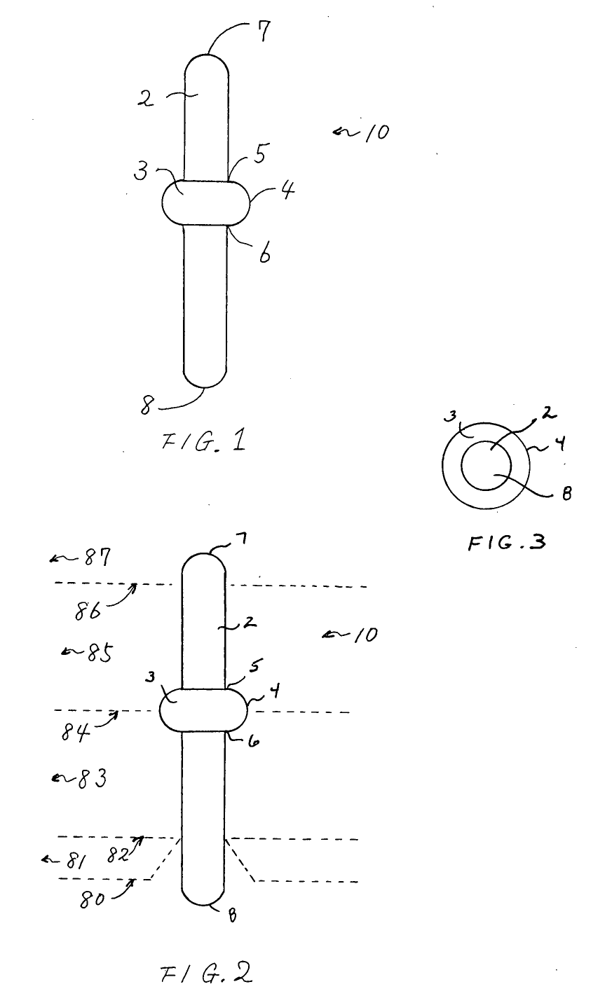

FIG. 1 shows the suppository 10 of the present invention suitable for use in hemorrhoid treatment. The suppository 10 of the present invention has a shaft 2 with a ring 3 formed thereon. The ring 3 is formed between ends 7 and 8 of the shaft 2. The shaft 2 has a cylindrical shape diameter between the ends 7 and 8. The ring 3 and the shaft 2 in the preferred embodiment, are formed of a medication material that is suitably dissolvable when received within the anal canal. The ring 3 and the shaft could also be of a solid polymeric material. The ring 3 extends outwardly of the shaft 2 and has a diameter that is greater than the diameter of the shaft 2.

The ring 3 has a generally toroidal shape. In other words, the outer periphery of the ring 3 is suitably curved so as to smoothly conform to the dentate of the anal canal. The top 5 and the bottom 6 of the ring 3 have a generally planar structure extending radially outwardly of the shaft 2. The ring 3 has a wide portion 4. This wide portion 4 has a diameter between 4 millimeters and 14 millimeters. The thickness of the ring 3 between the top 5 and the bottom 6 is no more than the wide portion 4 of the ring 3.

The top end 7 of the shaft 2 has a dome shape. Similarly, the bottom end 8 of the shaft 2 is also of a dome shape. The minimum length of the shaft 2 between the top end 7 and the bottom end 8 is 3 centimeters. In the preferred embodiment of the present invention, the ring 3 is integrally formed with the shaft 2. The minimum length from the wide portion 4 of the ring 3 to either end of the shaft is approximately 1.5 centimeters.

FIG. 2 illustrates the suppository 10 of the present invention within the anal canal. The broken line 80 illustrates the entrance of the anus. Area 81 is the external sphincter that contains the external hemorrhoids. Broken line 82 is illustrative of the intermuscular groove. Area 83 illustrates the pecten. This pecten is a narrow passage section of the anal canal. Broken line 84 illustrates the dentate line. This dentate line is a slightly indented portion in the anal canal.

Area 85 illustrates the internal hemorrhoid area. The broken line 86 indicates the upper edge of the internal hemorrhoid area. Area 87 is the ampulla of the rectum. The anal canal is between the intermuscular groove 82 and the upper edge 86 of the internal hemorrhoid area 85.

In a typical human body, the internal hemorrhoidal area 85 will have a length of between 18 and 20 millimeters. The pecten area 83 will have a length of between 15 and 20 millimeters. The external sphincter area 81 will have a length of between 5 and 10 millimeters. The anal canal is between the intermuscular groove line 82 and the upper edge 86 of the internal sphincter area. The length of the anal canal is typically between 30 millimeters and 40 millimeters inclusive.

As can be seen in FIG. 2, the suppository 10 is inserted into the anal canal such that the ring 3 of the suppository 10 fits into the dentate line 84. This is a balanced position of the suppository 10 within the anal canal. The suppository 10 will stay in this position. Even the strongest voluntary rectal pressure should not remove the suppository 10 from this position because the rectal pressure is not strong enough to push out the small ring 3 that is fit in the dentate line 84. Additionally, the relative friction between the surface of the shaft 2 and the wall of the anal canal is significant. As such, the suppository 10 will stay in the position until it dissolves. If the diameter of the shaft is more than 8 mm or the diameterr of the ring is more than 14 millimeters, then involuntary rectal and lateral pressure of the anal canal will imediately push out and dispose of the device 10 from the anus.

As can be seen in FIG. 3 in the preferred embodiment of the present invention, the ring 3 extends entirely around the outer diameter of the shaft 2. In this preferred embodiment, the bottom end 8 of the suppository 10 has a generally dome-like structure. Also in the preferred embodiment, the ring 3 has a generally constant diameter entirely around the shaft 2.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

1-2. (canceled)

3-4. (canceled)

5. A suppository for use in hemorrhoid treatment comprising:
a shaft having a first end and a second end, said shaft being generally cylindrical; and
a ring affixed on said shaft between said first end and said second end, said ring extending outwardly from said shaft, said ring having an outermost diameter of between 4 millimeters and 12 millimeters inclusive, said shaft having a length between said first end and said second end of no less than approximately 3 centimeters, said shaft having a diameter between ⅓and ⅔of said outermost diameter of said ring.
